(19) United States
(12) Patent Application Publication
(10) Pub. No.: US 2017/0225958 A1
(43) Pub. Date: Aug. 10, 2017

(54) FUNCTIONAL PARTICLES, PRODUCTION PROCESS AND USES

(71) Applicant: SMART INOVATION, LDA, Barcelos (PT)

(72) Inventors: César André DE SÁ MARTINS, Aldreu (PT); Afonso Henrique MARTINS INÁCIO, Barcelos (PT)

(21) Appl. No.: 15/501,010

(22) PCT Filed: Jul. 31, 2015
(86) PCT No.: PCT/IB2015/055834

§ 371 Date: Feb. 1, 2017

(30) Foreign Application Priority Data
Aug. 1, 2014 (PT) 107828

(51) Int. Cl. C01B 33/12; A01N 25/12; A61K 8/02; C01G 9/02; C11D 17/06; C11D 3/12; C11D 3/16; C09D 7/12; A61K 9/16; A61Q 19/00

(52) U.S. Cl. CPC C01B 33/12; A61K 9/1611; A01N 25/12; A61K 8/0275; A61Q 19/00; C11D 17/06; C11D 3/1213; C11D 3/162; C09D 7/1266; C09D 7/1275; C09D 7/1233; C01G 9/02; A61K 2800/412; A61K 2800/10

(57) ABSTRACT

The present disclosure relates to a process for obtaining and formulating functional silica particles and other materials with active ingredients/compounds for use in polymers, paints, mortars, ceramic, cement, textile, pharmaceutics, varnishes, paper, clays, cosmetics, sensors and effluents.

The present disclosure describes a functional particle for binding to a substrate comprising:

a granule comprising oxide or hydroxide of an element selected from the following list: silica, magnesium, zinc, iron, copper, silver, aluminum, gold, titanium, or mixtures thereof having a size between 90 nm-500 nm;

a binder comprising silane-based compounds which binds the outer granule to the substrate;

a functional compound/active compound bound to the surface of the granule, to the binder, or to both;

wherein the functional compound is at least one of the following compounds: anti-mosquito/repellent/anti-insect, fungicide, antimicrobial/bactericide, antimycotic, anti-fire, UV protectors, larvicides, hydrophobics, vitaminics, moisturizers, cosmetics, drugs, mechanical properties, magnetic properties enhancement, or mixtures thereof.

### TECHNICAL DOMAIN

The present disclosure relates to a process for obtaining and formulating functional silica particles and other materials with or without active ingredients for use in polymers, paints, mortars, ceramic, cement, textile, pharmaceutics, varnishes, paper, clays, cosmetics, sensors and effluents.

The active ingredients/functional compounds to be transported can be chosen from the following group: bactericides, fungicides, antimycotics, aromas, repellants, insecticides, pesticides, larvicides, vitamins, buffer solutions, moisturizers, cosmetics, drugs, UV protectors, anti-fire, increased mechanical properties, magnetic properties, hydrophobics and electrical conductors.

### BACKGROUND

WO2010114632A2 discloses a process for metal nanoparticle functionalization that allows them to have reversible aggregation/disaggregation properties. The composition is a nanoparticle functionalized with a coating material and the outer portion of the coating material is functionalized with polyethylene glycol (PEG).

US 2010/0136124 A1 discloses a process for drug release in the skin and describes a formulation within a particle-coated capsule meant to be applied to the skin, for example for the treatment of a disease. The described formulation comprises oil-based or aqueous droplets containing an active ingredient inside a nanoparticle coating, particularly silica nanoparticles.

WO2006102377A2 discloses a contrast process for use in Magnetic Resonance Imaging. It refers to magnetic nanoparticle functionalization with a functional group to be applied in the biomedical field, for example, in magnetic resonance imaging. The composition comprises: a functionalized magnetic nanoparticie, having a functional group with differential affinity with a tissue of the brain, and a pharmaceutically acceptable carrier.

These facts are described to illustrate the technical problem solved by the embodiments of the present document.

### GENERAL DESCRIPTION

The present disclosure describes a functional particle for binding to a substrate comprising:
a granule comprising oxide or hydroxide of an element selected from the following list: silica, magnesium, zinc, iron, copper, silver, aluminum, gold, titanium, or mixtures thereof having a size between 90 nm-500 nm;
a binder comprising silane-based compounds which binds the outer granule to the substrate;
a functional compound/active compound bound to the surface of the granule, to the binder, or to both;
wherein the functional compound is at least one of the following compounds: anti-mosquito/repellent/anti-insect, fungicide, antimicrobial/bactericide, antimycotic, anti-fire, UV protectors, larvicides, hydrophobics, vitamins, moisturizers, cosmetics, drugs, mechanical properties enhancement, magnetic properties, or mixtures thereof, among others.

The particles described are functionalized with several active ingredients, which are on the surface, and binding agents, but which do not involve a capsule.

The functional particles described in the present disclosure increase the effect of the active compound (or functional compound)—see FIGS. 6/7 and also manage for the effect to prevail even after several uses and or washes—see Tables I-VIII.

The granule does not have a fixed shape and can be spherical, oval, irregular, porous. The size of the granule can be obtained by various methods, such as sol-gel and simple salts precipitation.

The size of the granules and/or particles can be determined by several conventional methods including electron microscopy scanning electron microscope (SEM).

in an embodiment the particles described in the present disclosure have a particle size ranging from 90 nm-500 nm, preferably 120 nm-250 nm.

In an embodiment of the particles described in the resent disclosure, the substrate can be selected from the following list: polymers, paints, mortars, clays, cements, textiles, varnishes, paper, cosmetics, detergents, laminates, non-woven fabric, leathers, sensors and effluents.

In an embodiment of the particles described in the present disclosure, the functional compound/active compound is at least one of the following: anti-mosquito/repellent/anti-insect, fungicide, antimicrobial/bactericide, antimycotic, anti-fire, vitamins, moisturizers, dies, UV protectors, aromas, essential oils, drugs, larvicides, magnetics, hydrophobics, or mixtures thereof.

in an embodiment of the particles described in the present disclosure, the repellant can be selected from: ethyl butylacetylaminopropionate, permethrin, deltamethrin, p-3,8metano-diol, icaridin/saltidin, N,N-dimethyl-meta-toluarnide (DEET), citronella oil, methylneodecanamide, fipronil, empenthrin, d-allethrin, piperonyl butoxide, transfluthrin, prallethrin, biphenothrin, pyriproxyfen, imidacioprid, brodifacourn, tetramethrin, bromidaclopride, bromadiolone, bumatetralyl, blocumafene, bipermethrin, citriodiol or mixtures thereof.

In an embodiment of the particles described in the present disclosure, the fungicide can be selected from the following list: celery oil, chlorhexidine gluconate, berizalkoniurn chloride, zinc oxide, tea tree oil, octylisothiazolinone, di-n-decyidimethylammoniumchloride, phenoxyethanol, benzimidazole or mixtures thereof.

In an embodiment of the particles described in the present disclosure, the antimicrobial can be selected among: benzoyl peroxide, celery oil, chlorhexidine gluconate, benzalkonium chloride, zinc oxide, tea tree oil, ortho phenyl phenol, 2-benzyl-4-chlorophenol, cetyltrimethylamnmoniurn bromide, silver nitrate, zinc nitrate, copper nitrate, octylisothiazolinone, di-n-decyidimethylammoniumchloride, phenoxyethanol, chitosan or mixtures thereof.

In an embodiment of the particles described in the present disclosure, the antimycotic can be selected from a list comprised by: ketoconazole, terbinafine, sertaconazole, clotrinazole, fluconazole, itraconazole, fluconazole, nystatin, celery oil, chlorhexidine gluconate, benzalkonium chloride, octylisothiazolinone, di-n-decyldimethylammoniumchloride or mixtures thereof.

In an embodiment of the particles described in the present disclosure, the anti-fire can be selected from a list comprised by: zinc borate, magnesium hydroxide, aluminum hydroxide, boric acid, aluminum polyphosphate, ammonium polyphosphate or mixtures thereof.

In an embodiment of the particles described in the present disclosure, the UV protector can be selected from a list comprised by: 1H-Benzotriazole, drometrizole trisiloxane, p-aminobenzoic acid, cinoxate, dioxybenzone, homosalate, octocrylene, octyl methoxycinnamate, avobenzone, titanium oxide, zinc oxide, benzophenone-9, bemotrizinole, or mixtures thereof.

In an embodiment of the particles described in the present disclosure, the drug can be selected from: ivermectin, diclofenac, indomethacin, piroxicam, naproxen, allantoin, caffeine, hyaluronic acid, salicylic acid, hydrogen, piketoprofen, ammonium lactate, or mixtures thereof.

In an embodiment of the particles described in the present disclosure, the larvicide can be selected from a list comprised by: Methyl 4-hydroxybenzoate, temephos, diflubenzuron, novaluron, pyriproxyfen, diflubenzuron, methoprene or mixtures thereof.

In an embodiment the particles described in the present disclosure can further comprise vitamins, moisturizers, cosmetics, or mixtures thereof.

In an embodiment of the particles described in the present disclosure, wherein the binder is selected from a list comprised by (3-glycidyloxypropyl)trimethoxysilane, octyltriethoxysilane, octyltrimethoxysilane, propyltriethoxysilane, propyltrimethoxysilane, 3-aminopropyltriethoxysilane, 3-aminopropyltrimethoxysilane, HYDROSIL 2909, HYDROSIL 2627, 3-glycidyloxypropyltriethoxysilane, Dynasylan F 8815, 3-methacryloxypropyltrimethoxysilane, Dynasylan® HYDROSIL 2926, 3-aminopropyl-methylmethoxysilane, 3-[diethoxy(methyl)silyl]propan-1-amine, 3-glycidyloxypropylmethyldimethoxysilane, 3-glycidyloxypropylmethyldiethoxysilane or mixtures thereof.

Another aspect of the present disclosure describes a composition comprising the particles described in the present disclosure.

In an embodiment the composition described in this disclosure can include:
1%-50% (w/v) of a binder, preferably 5-50% (w/v), more preferably 20-50% (w/v);
0.1-95% (w/v) of an active compound/functional compound, preferably 50-80% (w/v);
0.1%-98% (w/v) of a granule comprising oxide or hydroxide selected from the following list: silica, magnesium, zinc, iron, copper, silver, aluminum, gold, titanium or mixtures thereof having a size between 90 nm-500 nm, preferably 0.1-50% (w/v), more preferably 0.1-20% (w/v).

In an embodiment the composition described in the present disclosure can further comprise a softener, dispersant, solvent, or mixtures thereof.

In an embodiment the composition described in the present disclosure can be used in medicine, particularly in the treatment of skin infections, dermatitis, wounds, ulcers including diabetic foot (in diapers, sanitary towels, socks or swabs).

Another aspect of the present disclosure shows the use of the functional particles described and/or composition to functionalize substrates and improve the effect of the active compound.

Another aspect of the present disclosure discloses articles comprising the particles and/or compositions described. In particular, the articles are: clothing, or footwear, or headwear, or mortar, or polymers, or ceramic articles, or clay or rubber articles, or polymers, or paints, or cement, or varnishes, or paper, or health, or sensors, or chemicals, or cosmetics or pharmaceuticals.

Another aspect of the present disclosure describes a method for obtaining granules comprising the following steps:
dissolving in water a salt, a nitrate, or sulphate, or chloride of an element selected from the following list: silica, magnesium, zinc, iron, copper, silver, aluminum, gold, titanium or mixtures thereof under stirring;
optionally adding 01-10 g of a surfactant to the previous solution;
adding an alkaline solution to precipitate the previous solution under stirring for 30 min-1.5 h and obtaining the granules;
adding to the granules previously obtained the selected binders and active ingredients.

An embodiment of the method described comprises dissolving in water 40 ml water, 6-30 g sodium metasilicate under stirring.

In an embodiment of the method described salts can be:
a pentahydrate sodium metasilicate; or
a nitrate of the elements selected from the following list: silica, magnesium, calcium, iron, copper, silver, zinc, aluminum, gold, titanium or mixtures thereof; or
a sulfate of the elements selected from the following list: silica, magnesium, calcium, iron, copper, silver, zinc, aluminum, titanium or mixtures thereof; or
a chloride of elements selected from the following list: silica, silver, calcium, magnesium, iron, zinc, copper, aluminum, gold, titanium, and mixtures thereof or mixtures thereof.

In an embodiment of the method described the surfactant is selected from the following list: polyether, polyethylene glycol (PEG), polyethylene oxide (PEO), polyethylene oxide-co-polypropylene oxide (PPO), co-polyethylene oxide, polyvinyl alcohol (PVA), poly(vinyl pyrrolidinone) (PVP), polyethylene oxide-polypropylene oxide-polyethylene-oxide (PEO-PPO-PEO), cetrimonium bromide, sodium dodecyl sulfate (SDS), or mixtures thereof.

In an embodiment of the method described alkaline or acidic solutions are used in the precipitation.

Another aspect of the present disclosure describes a method of application of the particles and/or compositions described in the preceding claims comprising spraying, dipping or aditivation of the functional particles and/or compositions herein described.

Throughout the specification and claims the word “comprising” and variations thereof, are not intended to exclude other technical features, additives, components, or steps. Additional objects, advantages and features of the disclosure will become apparent to those skilled in the art upon examination of the specification, or may be learned upon practice of the disclosure. The following examples and drawings are provided by way of illustration and are not intended to be limiting of the present disclosure. Furthermore, the present disclosure covers all possible combinations of particular or preferred embodiments herein described.

### DESCRIPTION OF THE DRAWINGS

For an easier understanding of the solution, drawings are herein attached, which represent preferred embodiments of the disclosure and which, however, are not intended to limit the scope of the present application.

FIG. 1: Representation of mosquito repellent silica particles in ceramic substrate.

FIG. 2: Representation of bactericide silica particles in textile substrate.

FIG. 3: Representation of aluminum particles in cement substrate.

FIG. 4: Representation of mite repellent silica particles in textile substrate.

FIG. 5: Representation of oxide particles in paper.

FIG. 6: Representation of dermatitis treatment with the application of the particles from example in Table III bactericidal in diapers; wherein Figure A represents the patient's skin after using conventional diapers and drugs, Figure B represents the patient's skin after using diapers with the particles described in Table III for 10 days; arid Figure C represents the patient's skin after using the diapers with the particles described in Table III for 20.

FIG. 7: Representation of ulcer treatment in diabetic foot with the application of the particles from example in Table III in conventional socks; wherein Figure A represents the patient's foot with the use of conventional socks and drugs, Figure B is represents the patient's skin after using socks with particles described in Table III for 1 month.

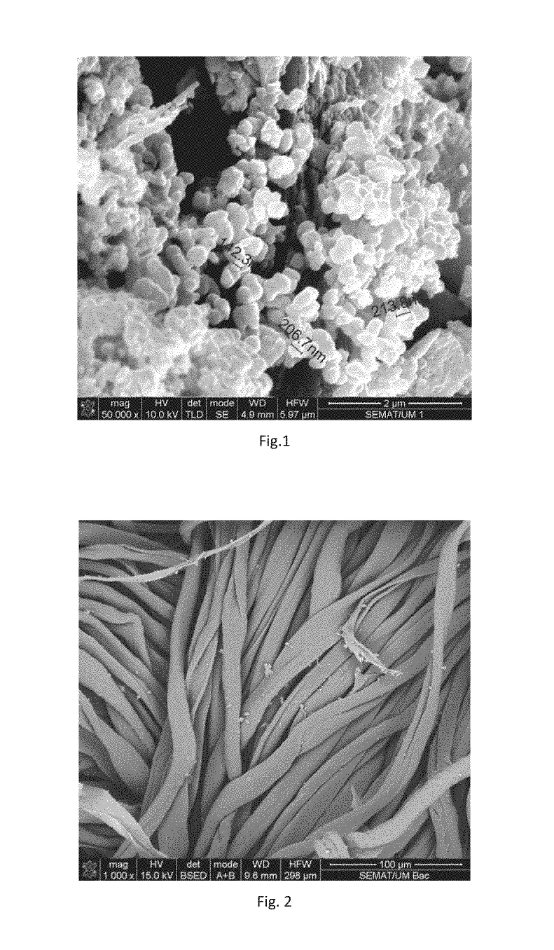

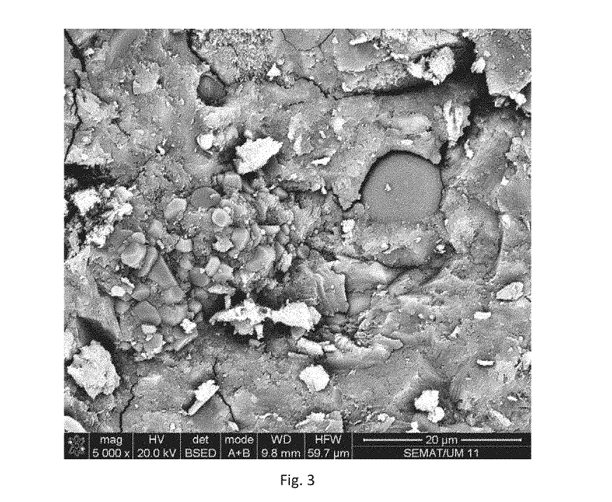

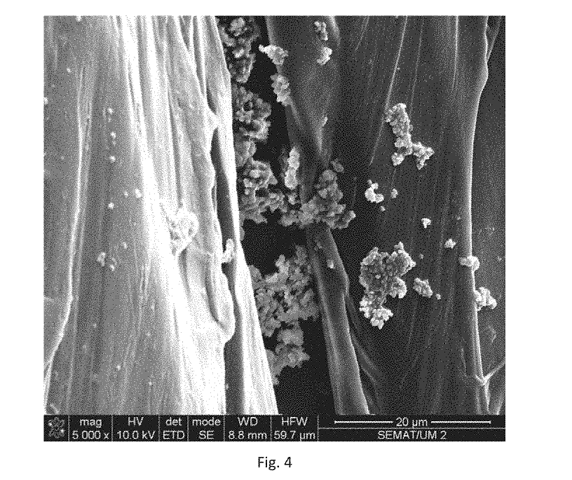

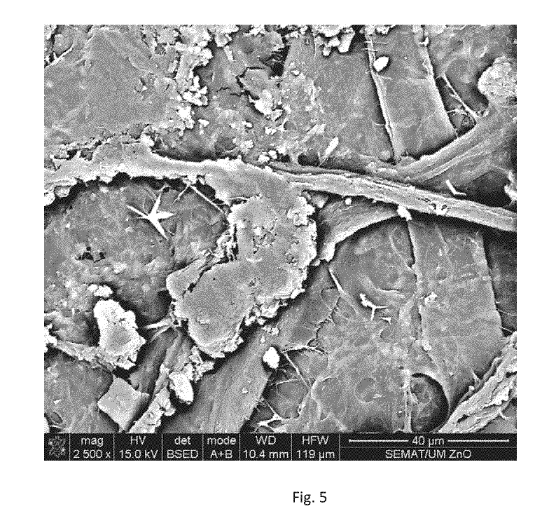

### DETAILED DESCRIPTION

The present disclosure relates to the process for obtaining functional particles with active ingredients and applications thereof in the textile field (as bactericidal, fungicidal, anti-odor, repellent, anti-mosquito, antimycotic, anti-fire, vitamins, moisturizers, pH control, absorbent, hydrophobic and drugs carrier), agricultural network (anti-insect). The present disclosure can also be used in mortars, cements, paints, varnishes, floors, pools (as a fungicide, repellant, larvicide, hydrophobic, for tensile strength and better frame connection), polymers (such as bactericidal, biodegradability, UV protection, gas impermeability, gas permeability, electrical conductivity, anti-fire, hydrophobic). Additionally it can be used for wastewater treatment, in pharmaceutical industry for drug delivery, paper (as bactericidal, fungicidal, anti-odor, repellent, antimycotic, anti-fire, hydrophobic, magnetic) and electronics.

The present disclosure can be used to functionalize the most varied materials in a simple application method with great efficiency and durability.

In an embodiment, the process for obtaining the 40 nm to 700 nm particles comprises the following steps:
a) Dissolving in 40 mL water 2 g-60 g, more preferably 6-30 g pentahydrate sodium metasilicate or nitrate of the elements selected from the following list (eg. calcium, iron, copper, silver, zinc, aluminum, gold, titanium, silica and mixtures thereof) or sulfate of elements selected from the following list (eg., magnesium, calcium, iron, copper, silver, zinc, aluminum, titanium, silica and mixtures thereof) or chloride of elements selected from the following list (eg. silver, calcium, magnesium, iron, zinc, copper, aluminum, gold, titanium, silica and mixtures thereof) under stirring;
b) Optionally adding, under stirring for 1-30 minutes, 0.1 g-10 g surfactant from the following list: Triton™ X100, Twenn® 20, 40, 60 and 80, polyether, polyethylene glycol (PEG), polyethylene oxide (PEO), polyethylene oxide-co-polypropylene oxide (PPO), polyvinyl alcohol (PVA), poly(vinyl pyrrolidinone) (PVP), bromide cetrimonium, sodium dodecyl sulfate (SDS), or mixtures thereof to the mixture resulting from step (a).
c) Adding 1-100 mL of active ingredient/functional compound or mixture of active ingredients/functional compounds, preferably able to be diluted with water, alcohols or solvents; even more preferably, in order to improve the bonding among the particles and active ingredient, 1-200 mL silane is added selected from the following group: (3-Glycidyloxypropyl)trimethoxysilane, octyltriethoxysilane, octyltrimethoxysilane, propyltriethoxysilane, propyltrimethoxysilane, 3-aminopropyltriethoxysilane, 3-aminopropyltriethoxysilane, HYDROSIL 2909, HYDROSIL 2627, 3-glycidyloxypropyltriethoxysilane, Dynasylan F 8815, 3-methacryloxypropyltrimethoxysilane, Dynasylan® HYDROSIL 2926, 3-aminopropyl-methyl-diethoxysilane, 3-[diethoxy(methyl)silyl]propan-1-amine, 3-glycidyloxypropylmethyldimethoxysilane, 3-glycidyloxypropylmethyldiethoxysilane or mixtures thereof.
d) Adding an alkaline solution (eg, sodium phosphate, ammonia, sodium hydroxide among other salts) for precipitating the above solution under stirring for 30 minutes to 1.5 hours but preferably 50-80 minutes, preferably a solution with 20 mL water with 1 g to 100 g ammonium chloride.

In an embodiment, the obtention process can be performed only with steps a) and d) obtaining particles for mixing with binders and active ingredients.

In some cases, depending on the application, the particles can be dried or filtered.

In the present disclosure the active ingredient can be selected from the following group:
repellents such as ethyl butylacetylaminopropionate, permethrin, deltamethrin, p-3,8metano-diol, icaridin/saltidin, N,N-dimethyl-meta-toluamide (DEFT), citronella oil, methylneodecanamide, fipronil, empenthrin, d-allethrin, piperonyl butoxide, transfluthrin, prallethrin, biphenothrin, pyriproxyfen, imidacloprid, brodifacoum, tetramethrin, bmidaclopride, bromadiolone, bumatetralyl, blocumafene, bipermethrin, citriodiol or mixtures thereof;
fungicides such as celery oil, chlorhexidine gluconate, benzalkonium chloride, zinc oxide, tea tree oil, octylisothiazolinone, di-n-decyldimethylammoniumchloride, phenoxyethanol, benzimidazole or mixtures thereof;
antimicrobial, such as benzoyl peroxide, celery oil, chlorhexidine gluconate, benzalkonium chloride, zinc oxide, tea tree oil, ortho phenyl phenol, 2-benzy-4-chlorophenol, cetyltrimethylammonium bromide, silver nitrate, zinc nitrate, copper nitrate, octylisothiazolinone, di-n-decyldimethylammoniumchoride, phenoxyethanol, chitosan or mixtures thereof;
antimycotics such as ketoconazole, terbinafine, sertaconazole, clotrinazole, fluconazole, itraconazole, fluconazole, nystatin, celery oil, chlorhexidine gluconate, benzalkonium chloride, octylisothiazolinone, di-n-decyldimethylammoniumchloride or mixtures thereof;
anti-fire such as zinc borate, magnesium hydroxide, aluminum hydroxide, boric acid, aluminum polyphosphate, ammonium polyphosphate or mixtures thereof; vitamins, moisturizers, dies;
UV protectors such as 1H-Benzotriazole, drometrizole trisiloxane, p-aminobenzoic acid, cinoxate, dioxybenzone, homosalate, octocrylene, octyl methoxycinnamate, avobenzone, titanium oxide, zinc oxide, benzophenone-9, bemotrizinole, or mixtures thereof;
flavorings, essential oils such as celery, mint, orange, apple, green tea, aloe vera, eucalyptus, lavender, lemon, DL-menthol, or mixtures thereof; drugs such as ivermectin, diclofenac, indomethacin, piroxicam, naproxen, allantoin, caffeine, hyaluronic acid, salicylic acid, hydrogen, piketoprofen, ammonium lactate, or mixtures thereof;
larvicides such as Methyl 4-hydroxybenzoate, temephos, diflubenzuron, novaluron, pyriproxyfen, diflubenzuron, methoprene or mixtures thereof;
anti-insects such as methylneodecanamide, fipronil, empenthrin, d-allethrin, piperonyl butoxide, transfluthrin, prallethrin, cyphenothrin, pyriproxyfen, imidacloprid, brodifacoum, tetramethrin, imidacloprid, bromadiolone, coumatetralyl, flocournafen, or mixtures thereof;

These active ingredients can be mixed in active ingredient/binder group ratios (w/v) of 10/90 or 20/80 or 30/70 or 40/60 or 50/50 or 60/40 or 70/30 or 80/20 or 90/10, and this concentrate can also be diluted in solvents, for example, in water or alcohols w/v at 10/90 or 20/80 or 30/70 or 40/60 or 50/50 or 60/40 or 70/30 or 80/20 or 90/10.

The functionalized particles and/or compositions described can be sprayed onto the surfaces of various materials.

The functionalized particles and/or compositions described can this solution be further mixed with paints, mortars, cements, textiles, pharmaceuticals, varnishes, electronics, polymers, sensors, civil construction, laminates, paper, paper pulp, diapers, swabs, sanitary towels, detergents, among others.

Effect of application of the particles described in the present disclosure for several applications, add 1% to 50% w/v.

Table I—Shows tests conducted with Aedis Egypti mosquitos using the particles herein described, namely with silica octyltriethoxysilane/ethyl butylacetylaminopropionate:

| Sample | % Repellency |
|---|---|
| Textile without particles | 0% |
| Textile with particles described, 0 washings | 100% |
| Textile with particles described, 50 washings | 91% |
| Textile with particles described, 80 washings | 84% |
| Textile with particles described, 100 washings | 75% |

Table II—Shows tests conducted with Aedis Egypti mosquitos using the particles herein described, namely with silica/3-aminopropyltriethoxysilanelethyl butylacetylaminopropionate:

| Sample | % Repellency |
|---|---|
| Paint with no particles described | 0% |
| Paint with particles described | 99.99% |

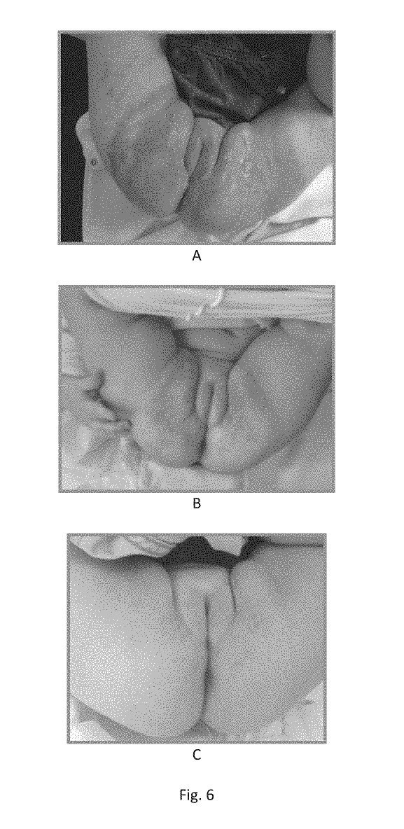

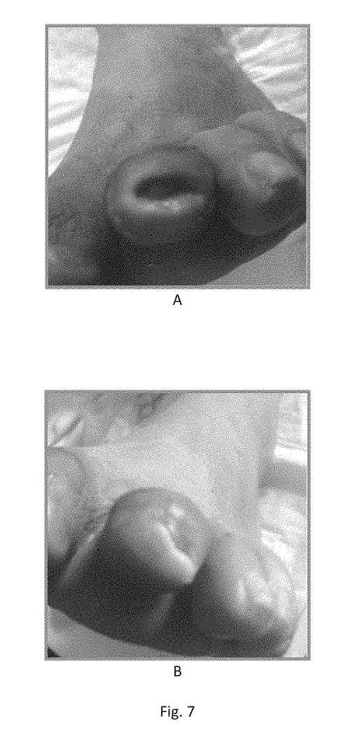

Table III—Shows tests performed with Staphylococcus aureus in diapers and socks using the particles herein described, particularly with silica/organofunctional siloxane oligomer reactive in water/benzalkonium chloride (see effect in FIGS. 6/7); in these cases, particles were applied by spraying (spray) in conventional diapers and socks:

| Sample | % Effectiveness |
|---|---|
| Textile without particles | 0% |
| Textile with particles described, 20 washings | 91% |
| Textile with particles described, 50 washings | 70% |

TABLE IV
Tests conducted with Staphylococcus aureus using the particles herein
described, in particular silica/organofunctional siloxane oligomer
reactive in water/chlorhexidine gluconate/benzalkonium chloride:

| Sample | % Effectiveness |
|---|---|
| Paint with no particles described | 0% |
| Paint with particles described | 99.99% |

TABLE V
Tests conducted with Aspergilius niger using the particles herein
described in conventional diapers and conventional socks, in particular
silica/organofunctional siloxane oligomer reactive in water/chlorhexidine
gluconate/benzalkonium chloride (see effect in FIGS. 6/7):

| Sample | % Effectiveness |
|---|---|
| Paint with no particles described | 0% |
| Paint with particles described | 99.99% |

TABLE VI
Tests conducted with Dermatophagoides pteronyssinus mite using the
particles herein described in particular silica/octyltriethoxysilane/Ethyl
Butylacetylaminopropionate:

| Sample | % Repellency |
|---|---|
| Textile without particles | 0% |
| Textile with particles described | 92.25% |

TABLE VII
Tests conducted with Dermatophagoides farinae mite using the particles
herein described in particular silica/octyltriethoxysilane/Ethyl
Butylacetylaminopropionate:

| Sample | % Repellency |
|---|---|
| Textile without particles | 0% |
| Textile with particles described | 85.8% |

TABLE VIII
Tests conducted with Staphylococcus aureus using the particles herein
described, in particular silica/organofunctional siloxane oligomer
reactive in water/benzalkonium chloride:

| Sample | % Effectiveness |
|---|---|
| Paper with no particles described | 0% |
| Paper with particles described | 99.99% |

Despite several washes which the material is subjected to, it has been found that functionality is maintained after such several washes, these properties being due to the structure of the particles, obtention process, composition and obtention method thereof.

### SYNTHESIS EXAMPLES

### Example 1

### Silica Particles

1g to 20 g pentahydrate sodium metasilicate is added to 40 mL water and dissolved so as to obtain an emulsion.

For the precipitation of the emulation, a 20 mL water side solution is added and 1 g to 50 g ammonium chloride, then the above solution is added and left under stirring for 5 minutes to 120 minutes.

### Example 2

### Synthesis of Zinc Hydroxide Particles

In 40 mL water 1 g to 200 g zinc nitrate or zinc sulfate is dissolved and stirred to complete dissolution.

Then, 0.1 g to 5 g Triton X100 is added and stirred for 1 to 30 minutes.

For the precipitation of the emulation, a 20 mL water side solution is prepared and 1 g to 50 g ammonium chloride or 5 mL to 100 mL 50% NaOH is added, then the previous solution is added and left under stirring for 5 minutes to 120 minutes.

### Example 3

### Synthesis of Silica Particles With Other Materials

1 g to 20 g pentahydrate sodium metasilicate is added to 40 mL water and dissolved.

Then the above solution is added with a 10 mL to 100 mL water solution with 1 g to 100 g nitrate of elements selected from the following list (eg. magnesium, calcium, iron, copper, silver, zinc, aluminum, gold, titanium, and mixtures thereof) or sulfate of elements selected from the following list (e.g., magnesium, calcium, iron, copper, silver, zinc, aluminum, titanium, and mixtures thereof) or chloride of elements selected from the following list (e.g. silver, calcium, magnesium, iron, zinc, copper, aluminum, gold, titanium, and mixtures thereof).

For the precipitation of the emulation, a 20 mL water side solution is prepared and 1 g to 50 g ammonium chloride or 5 mL to 100 mL 50% NaOH is added, then the previous solution is added and left under stirring for 5 minutes to 120 minutes.

### Example 4

### Synthesis of Antimycotic Silica Particles

1 g to 20 g pentahydrate sodium metasilicate is added to 40 mL water and dissolved.

Then the above solution 1 to 100 g ketoconazole and 1 to 10 mL (3-Glycidyloxypropyl)trimethoxysilane is added.

For the precipitation of the emulation, a 20 mL water side solution is prepared and 1 g to 50 g ammonium chloride is added and the previous solution is added and left under stirring for 5 minutes to 120 minutes.

### Example 5

### Active Ingredient

(3-Glycidyloxypropyl)trimethoxysiiane, octyltriethoxysilane, octyltrimeth-oxysilane, propyltriethoxysilane, propyltrimethoxysilane, 3-aminopropyltriethoxysilane, 3-aminopropyltrimethoxysilane, HYDROSIL 2909, HYDROSIL 2627, (3-glycidyloxy)propyltriethoxysilane, Dynasyian F 8815, 3-methacryloxypropyltrimethoxysilane, Dynasylan® HYDROSIL 2926, 3-aminopropyl-methyl-diethoxysilane, 3-[diethoxy(methyl)silyl]propan-1-amine, 3-Glycidyloxypropyl-methyldimethoxysilane, 3-Glycidyloxypropylmethyldiethoxysilane or mixtures thereof with Ethyl Butylacetylaminopropionate, among other active ingredients are mixed in a w/v ratio ranging 10/90 or 20/80 or 30/70 or 40/60 or 50/50 or 60/40 or 70/30 or 80/20 or 90/10, the concentrate can also be diluted in water, alcohols or solvents at a w/v ratio ranging 10/90 or 20/80 or 30/70 or 40/60 or 50/50 or 60/40 or 70/30 or 80/20 or 90/10.

All these Examples can be Applied:

The application of these particles can be applied by spraying or dipping or aditivation.

Dispersing or dry mixture with polymers, paints, mortars, cements, paper, paper pulp, detergents, textile, varnishes, ceramics, cosmetics, electronics, sensors, glass, civil construction, laminates, diapers, sanitary towels, swabs, medical devices, pharmaceutical articles, among others.

Example Application in Textile Stretching, Tumbler or Washing Machine:

Amount in examples 1,2,3,4,5: from 1 g/L to 200 g/L

Binder: 1 g/L to 300 g/L (e.g. ((3-Glycidyloxypropyl)trimethoxysilane, octyltriethoxysilane, octyltrimethoxysilane, propyltriethoxysilane, propyltrimethoxysilane, aminopropyltriethoxysilane, 3-aminopropyltrimethoxysilane, HYDROSIL 2909, HYDROSIL 2627, 3-glycidyloxypropyitriethoxysilane, Dynasylan F 8815, 3-methacryloxypropyltrimethoxysilane, Dynasylan® HYDROSIL 2926, 3-aminopropyl-methyl-diethoxysilane, 3-[diethoxy(methyl)silyl]propan-1-amine, 3-glycidyloxypropylmethyldimethoxysilane, 3-glycidyloxypropylmethyldiethoxysilane or mixtures thereof).

Drying is carried out between 20° C. to 200° C.

### Application Example in Paints and Varnishes:

Amount in examples 1,2,3,4,5: between 1 g/L to 300 g/L, mix and apply paint or varnish.

The embodiments described above are combinable.

The present disclosure is of course in no way restricted to the embodiments herein described and a person of ordinary skill in the art will be able to provide many modification possibilities thereto and substitutions of technical features for equivalents, according to requirements in each situation, as defined in the claims.

The following claims further define preferred embodiments.

1-22. (canceled)

23. A functional particle for binding to a substrate, comprising:
0.1-50% p/v of a granule comprising oxide or hydroxide of an element having a size between 90 nm-500 nm and being selected from the following group consisting of: silica, magnesium, zinc, iron, copper, silver, aluminum, gold, titanium, and mixtures thereof
5-50% (p/v) of a binder comprising silane-based compounds that bind the outer granule to the substrate; and
40-80% (p/v) of a functional compound/active compound bound to the surface of the granule, to the binder, or to both;
wherein the functional compound/active compound comprises at least one of the following compounds: anti-mosquito/repellent/anti-insects, fungicide, antimicrobial/bactericide, antimycotic, anti-fire, UV protectors, larvicides, hydrophobics, vitaminics, moisturizers, cosmetics, drugs, and mixtures thereof.
wherein the particle size ranges is between 120 nm-250 nm.

24. The particle according to claim 23, comprising:
20%-50% (w/v) of a binder;
50-80% (w/v) of an active compound/functional compound;
0.1%-20% (w/v) of a granule comprising oxide or hydroxide of an element having a size between 90 nm-500 nm and being selected from the following the group consisting of: silica, magnesium, zinc, iron, copper, silver, aluminum, gold, titanium. and mixtures thereof.

25. The particle according to claim 23, wherein the binder is selected from the group consisting of: (3-glycidyloxypropyl) trimethoxysilane, octyltriethoxysilane, octyltrimeth-oxysilane, propyltriethoxysilane, propyltrimethoxysilane, 3-aminopropyltriethoxysilane, 3-aminopropyltrimethoxysilane, organofunctional oligosiloxane reactive in water, fluoroalilfunctional oligosiloxane, 3-glycidyloxypropyltriethoxysilane, 3-methacryloxypropyltrimethoxysilane, 3-aminopropyl-methyl-diethoxysilane, 3-[diethoxy(methyl)silyl]propan-1-amine, 3-glycidyloxypropylmethyldimethoxysilane, 3 -glycidyloxypropylmethyldiethoxysilane, and mixtures thereof.

26. The particle according to claim 23, wherein the binder is selected from the group consisting of: (3-glycidyloxypropyl) trimethoxysilane, octyltriethoxysilane, 3-aminopropyltriethoxysilane, organofunctional oligosiloxane reactive in water, fluoroalilfunctional oligosiloxane, 3-aminopropyl-methyl-diethoxysilane, 3-[diethoxy(methyl)silyl]propan-1-amine, 3-glycidyloxypropylmethyldimethoxysilane, 3-glycidyloxypropylmethyldiethoxysilane, and mixtures thereof.

27. The particle according to claim 23, wherein the functional compound/active compound comprises the repellant and wherein the repellant is selected from the group consisting of: ethyl butylacetylaminopropionate, permethrin, deltamethrin, p-3,8metano-diol, icaridin/saltidin, N,N-dimethyl-meta-toluamide, citronella oil, methylneodecanamide, fipronil, empenthrin, d-allethrin, piperonyl butoxide, transfluthrin, prallethrin, biphenothrin, pyriproxyfen, imidacloprid, brodifacoum, tetramethrin, bmidaclopride, bromadiolone, bumatetralyl, blocumafene, bipermethrin, citriodiol, and mixtures thereof.

28. The particle according to claim 23, wherein the functional compound/active compound comprises the fungicide and wherein the fungicide is selected from the group consisting of: celery oil, chlorhexidine gluconate, benzalkonium chloride, zinc oxide, tea tree oil, octylisothiazolinone, di-n-decyldimethylammoniumchloride, phenoxyethanol, benzimidazole, and mixtures thereof.

29. The particle according to claim 23, wherein the functional compound/active compound comprises the antimicrobial and wherein the antimicrobial is selected from the group consisting of: benzoyl peroxide, celery oil, chlorhexidine gluconate, benzalkonium chloride, zinc oxide, tea tree oil, ortho phenyl phenol, 2-benzyl-4 -chlorophenol, cetyltrimethylammonium bromide, silver nitrate, zinc nitrate, copper nitrate, octylisothiazolinone, di-n-decyldimethylammoniumchloride, phenoxyethanol, chitosan, and mixtures thereof.

30. The particle according to claim 23, wherein the functional compound/active compound comprises the antimycotic and wherein the antimycotic is selected from the group consisting of: ketoconazole, terbinafine, sertaconazole, clotrinazol, fluconazole, itraconazole, fluconazole, nystatin, celery oil, chlorhexidine gluconate, benzalkonium chloride, octylisothiazolinone, di-n-decyldimethylammoniumchloride, and mixtures thereof.

31. The particle according to claim 23, wherein the functional compound/active compound comprises the anti-fire and wherein the anti-fire is selected from the group consisting of: zinc borate, magnesium hydroxide, aluminum hydroxide, boric acid, aluminum polyphosphate, ammonium polyphosphate, and mixtures thereof.

32. The particle according to claim 23, wherein the functional compound/active compound comprises the UV protector and the wherein the UV protector is selected from the group consisting of: 1H-Benzotriazole, drometrizole trisiloxane, p-aminobenzoic acid, cinoxate, dioxybenzone, homosalate, octocrylene, octyl methoxycinnamate, avobenzone, titanium oxide, zinc oxide, benzophenone-9, bemotrizinole, and mixtures thereof.

33. The particle according to claim 23, wherein the functional compound/active compound comprises a drug and wherein the drug is selected from the group consisting of: ivermectin, diclofenac, indomethacin, piroxicam, naproxen, allantoin, caffeine, hyaluronic acid, salicylic acid, piketoprofen, ammonium lactate, and mixtures thereof.

34. The particle according to claim 23, wherein the functional compound/active compound comprises the larvicide and the preceding claims wherein the larvicide is selected from the group consisting of: Methyl 4-hydroxybenzoate, temephos, diflubenzuron, novaluron, pyriproxyfen, diflubenzuron, methoprene, and mixtures thereof.

35. The particle according to claim 23, further comprising vitamins, moisturizers, cosmetics, essential oils, or mixtures thereof

36. A composition comprising functional particles configured to bind to a substrate, the particles comprising:
0.1-50% p/v of a granule comprising oxide or hydroxide of an element having a size between 90 nm-500 nm and being selected from the following group consisting of: silica, magnesium, zinc, iron, copper, silver, aluminum, gold, titanium, and mixtures thereof;
5-50% (p/v) of a binder comprising silane-based compounds that bind the outer granule to the substrate; and
40-80% (p/v) of a functional compound/active compound bound to the surface of the granule, to the binder, or to both;
wherein the functional compound/active compound comprises at least one of the following compounds: anti-mosquito/repellent/anti-insects, fungicide, antimicrobial/bactericide, antimycotic, anti-fire, UV protectors, larvicides, hydrophobics, vitaminics, moisturizers, cosmetics, drugs, and mixtures thereof.
wherein the particle size ranges is between 120 nm-250 nm.

37. The composition according to claim 36, further comprising a softener, a dispersant, solvent, or mixtures thereof.

38. A method for the treatment of skin infections including dermatitis, wounds, ulcers, namely diabetic foot, comprising applying a composition comprising the functional particles of claim 36.
